(19) United States
(12) Patent Application Publication
(10) Pub. No.: US 2007/0106263 A1
(43) Pub. Date: May 10, 2007

(54) Intravenous identification luminaire (IV-ID) and light pipe, and light conductive intravenous delivery system

(76) Inventor: Russell Craig Ward, Sun Prairie, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/257,335

(22) Filed: Oct. 24, 2005

(51) Int. Cl. A61N 1/30; A61M 39/10; A61M 39/00; A61M 25/16; A61M 25/18

(52) U.S. Cl. 604/533; 604/21

(57) ABSTRACT

An intravenous identification luminaire (IV-ID) and light pipe, and a light conductive intravenous delivery system is described. The light pipe preferably includes a female and male luer fitting, a fluid port, and a light source socket such that the light pipe is configured to be spliced into an IV line, and the IV-ID is coupled to the light pipe. The light pipe allows normal flow of the prescribed fluid through the light pipe while conducting light from the IV-ID, through the light source socket and along a conductive light path. The conductive light path continues into the IV line. The system also includes an indicator panel at the distal end of the IV line configured to reflect and make obvious the color of that particular IV line to a user.

### FIELD OF THE INVENTION

The invention relates to the field of intravenous (IV) drug administration. More particularly, the invention relates to the identification of individual IV lines.

### BACKGROUND OF THE INVENTION

Patients in emergency, critical care, cardiac and operating room situations are almost without exception treated with fluid and drug delivery regimens that require single or multiple intravenous (IV) access lines to deliver the prescribed fluid and drug treatment. In some extreme situations, the number of IV lines can, for a given patient, exceed 32 separate IV lines. These IV lines may be routed through infusion pumps or simply connected as stand alone gravity based IV lines. This fluid and drug therapy regimen can therefore create a complex web of these IV lines.

An implication of this web is the importance of managing drugs and fluids to ensure that incompatible drugs and fluids are not mixed together. This problem can be described as an IV line identification management issue that is ordinarily managed by the clinician visually identifying each IV line and literally following the IV by hand to confirm the right line has been chosen. This process is time consuming and thwarted with potential error. Incorrect drug delivery into the wrong line can create serious and potentially dangerous implications for the patient. What is needed is clinician assistance in identifying IV lines in this situation.

### SUMMARY OF THE INVENTION

An intravenous identification luminaire (IV-ID) and light pipe, and a light conductive intravenous delivery system is described. The light pipe preferably includes a female and male luer fitting, a fluid port, and a light source socket such that the light pipe is configured to be spliced into an IV line, and the IV-ID is coupled to the light pipe. The light pipe allows normal flow of the prescribed fluid through the light pipe while conducting light from the IV-ID, through the light source socket and along a conductive light path. The conductive light path continues into the IV line. The system also includes an indicator panel at the distal end of the IV line configured to reflect and make obvious the color of that particular IV line to a user.

A light pipe for conducting light from an identification luminaire to an intravenous line comprising a fluid port configured to be spliced into the intravenous line, wherein the fluid port allows continuous flow of a fluid through the intravenous line, a light conducting medium configured to define the fluid port, a first fitting and a second fitting configured on a first end and a second end of the fluid port, such that the fittings effectuate connection to the intravenous line, and a light source connection socket configured to removably house the identification luminaire, and further configured to channel light from the identification luminaire into the light conducting medium, wherein the light conducting medium is configured to conduct the light into the intravenous line. The light pipe, wherein the first fitting and the second fitting are a luer fitting, wherein the intravenous line is coated with a translucent coating, wherein the light pipe is coated with a translucent coating, wherein the identification luminaire emits colored light, and wherein the identification luminaire is a light emitting diode. The light pipe, further comprising an indicator panel configured on a distal end of the intravenous line, wherein the indicator panel is a reflective material, configured to reflect the light in the light conducting medium out of the light conducting medium, wherein the indicator panel is affixed to the outside surface of the intravenous line, and wherein the indicator panel is embedded in the intravenous line.

The light pipe, wherein the indicator panel includes a housing configured to enclose a portion of the intravenous line and an extension of the light conducting medium, such that the extension is in contact with the intravenous line and the housing, wherein the housing includes a transparent portion configured to allow the conducted light to leave the housing.

A light conductive IV system comprising an intravenous line configured to deliver a fluid from a fluid container to a patient, a light pipe inserted along the path of the intravenous line and including a fluid port configured to allow continuous flow of the fluid through the intravenous line, wherein a light conducting medium defines the fluid port, an identification luminaire coupled to the light pipe, wherein the light pipe channels light from the identification luminaire into the light conducting medium, wherein the light conducting medium is configured to conduct the light into the intravenous line, and an indicator panel configured on a distal end of the intravenous line, wherein the indicator panel is a reflective material, configured to reflect the light in the light conducting medium out of the light conducting medium. The light conductive IV system, further including a first fitting and a second fitting configured on a first end and a second end of the fluid port, such that the fittings effectuate connection to the intravenous lines, wherein the first fitting and the second fitting are a luer fitting, wherein the light pipe is molded into the intravenous line in a permanent fashion, and wherein the intravenous line and the light pipe are coated with a translucent coating. The light conductive IV system, wherein the identification luminaire emits colored light, and further wherein when the system includes a plurality of intravenous lines, a plurality of identification luminaires are utilized, each identification luminaire emitting a different color light. The light conductive IV system, wherein the identification luminaire is a light emitting diode, wherein the indicator panel is affixed to the outside surface of the intravenous line, and wherein the indicator panel is embedded in the intravenous line.

A light pipe comprising a fluid port configured to be spliced into an intravenous line, wherein the fluid port allows continuous flow of a fluid through the intravenous line, a light conducting medium configured to define the fluid port, and further configured to allow continuous light conduction through the intravenous line, a first luer fitting and a second luer fitting configured on a first end and a second end of the fluid port, such that the luer fittings effectuate connection to the intravenous line, and a light source connection socket configured to removably house an identification luminaire, and further configured to channel light from the identification luminaire into the light conducting medium, wherein the light conducting medium is configured to conduct the light into the intravenous line, and further wherein the light pipe is coated with a translucent coating.

### BRIEF DESCRIPTION OF THE DRAWINGS

FIG. 1 is a graphical representation of a light pipe according to an embodiment of the present invention.

FIG. 2 illustrates a block diagram illustrating a system of an embodiment of the present invention.

FIGS. 3A-3C are graphical representations illustrating various embodiments of an indicator panel of the present invention.

### DETAILED DESCRIPTION OF THE INVENTION

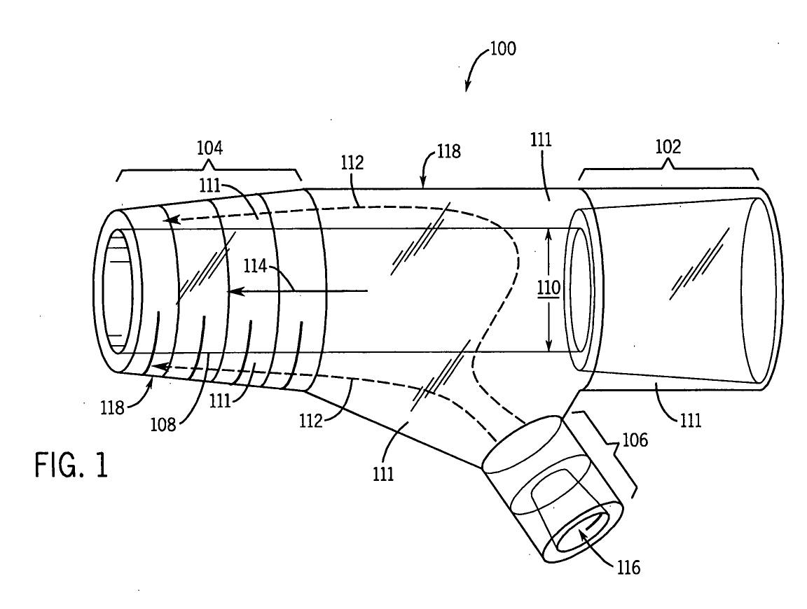

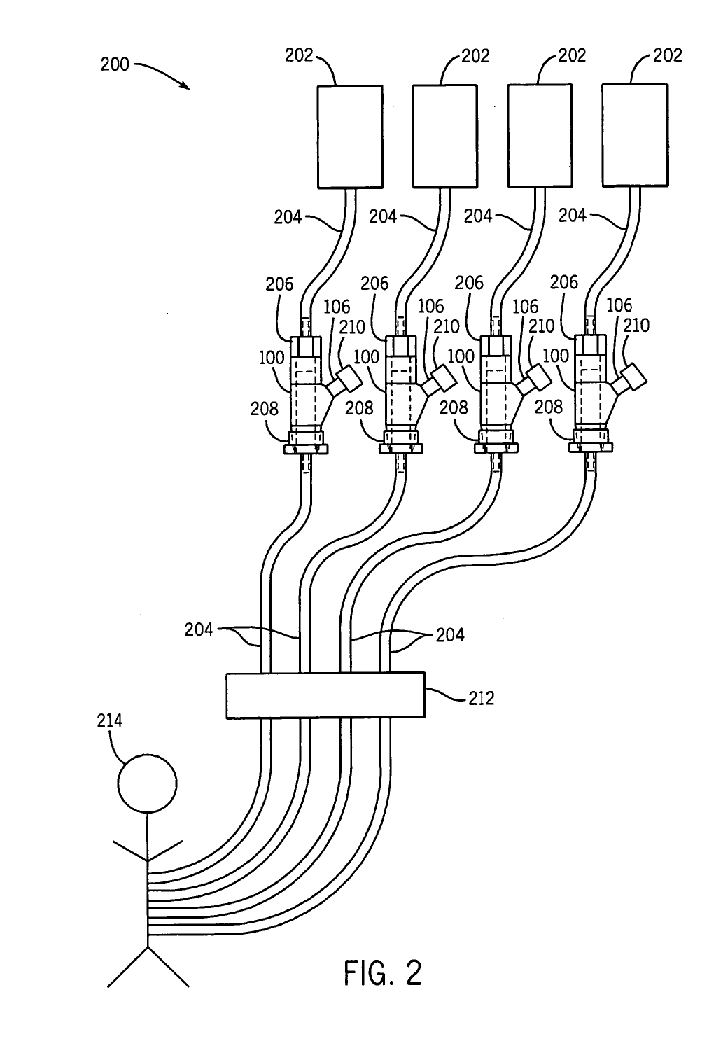

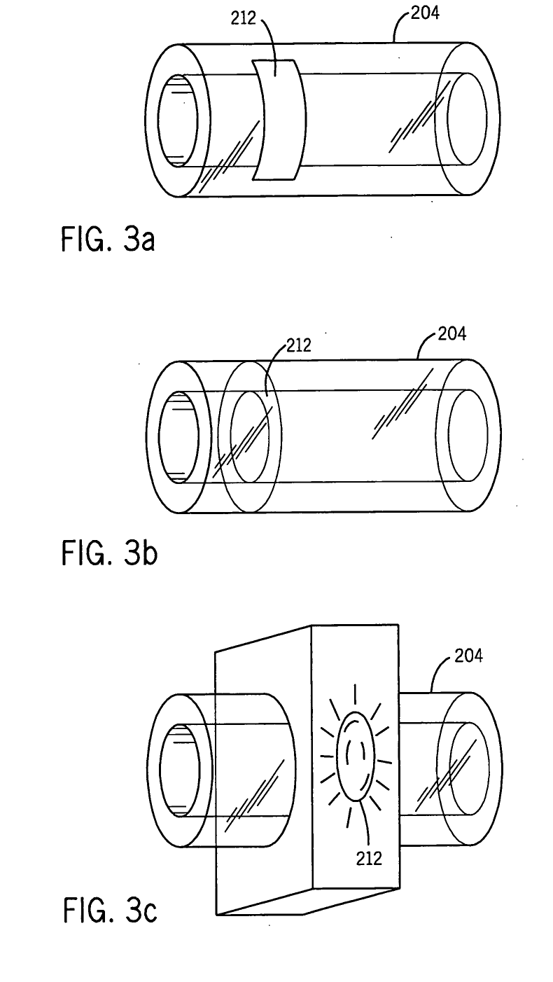

In FIG. 1, the intravenous identification luminaire (IV-ID) is a light emitting diode (LED) device, or a similar light source that is projected into a light pipe 100. The light pipe 100 is connected to a compatible IV line and is preferably positioned proximally to the fluid source and upstream of IV junctions and IV line manifolds. The light emitted from the IV-ID is focused by the light pipe 100 and reflected into the plastic hose body of the IV line to illuminate the IV line such that it can be clearly identified along the hose line length from other non-illuminated lines, or lines illuminated with a different color. The light pipe 100 design is intentionally designed to focus the IV-ID light into the wall of the compatible IV line to “pipe” the light to an indicator panel 212 (FIG. 2-3C) on the distal end of the compatible IV line. When there is a need to track multiple lines, multiple IV-ID's can be used that emit different colors.

The light conductive intravenous delivery system of the present invention is specifically designed to incorporate the IV-ID and light pipe 100 to facilitate the incidence of light emitted from the IV-ID along the length of the IV delivery system. This design conducts the projected light to an indicator panel 212 on the distal end of the IV line. A translucent coating on the outside of the IV line, as well as on the outside of the light pipe, reduces loss of the light from the hose and system.

FIG. 1 depicts a light pipe 100 of an embodiment of the present invention. The light pipe 100 includes a fluid port 108 having a diameter 110 of preferably 3 millimeters. The internal diameter may be configured to fit any size IV line. The prescribed fluid preferably moves through the light pipe 100 as shown by the fluid path 114. The light pipe 100 is fashioned such that it may be spliced into an existing IV line, on the end of the IV line proximate to the prescribed fluid source. Preferably, the light pipe 100 includes a female luer fitting 102 and a male luer fitting 104, such that the appropriate luer lock fittings may be coupled to the light pipe 100 and spliced into the IV line. Of course, any method known in the art may be utilized to insert the light pipe 100 into the IV line. It is contemplated that one such solution may include permanently molding the light pipe 100 into the IV line.

Still referring to FIG. 1, a light conducting medium 111 surrounds the fluid port 108, and preferably the female luer fitting 102 and the male luer fitting 104 are also fashioned from light conducting medium. Preferably, the light conducting medium 111 is the same material as the IV line. However, any light conducting medium known in the art may be utilized in the light pipe 100. A light source socket 106 is fashioned on the outer surface of the light pipe 100 and provides an IV-ID entrance 116 to the light conducting medium 111, such that the IV-ID may be removably coupled to the light pipe 100, thus providing a light source along a conducted light path 112. The IV-ID is preferably a light emitting diode (LED), but may be any light source appropriate to provide a light source to the light pipe 100.

The light conducting medium 111 of the light pipe 100 will conduct the conductive light path 112 through the light pipe, as pictured in FIG. 1, and into the light conducting medium 111 of the IV line. In order to limit the amount of light escaping through reflection from the light pipe 100 and the IV line, the light pipe surface 118, as well as the surface of the IV line may be coated with a translucent material that will reflect some of the light back into the light pipe 100, while allowing a portion of the light to leave the light pipe 100 for identification purposes.

Referring now to FIG. 2, a light conductive IV system 200 of the present invention is depicted. Prescribed fluid 202, including prescription medication, saline, or other prescribed fluids, are administered through an IV line 204 to the light pipe 100. Once again, the light pipe 100 is spliced into the IV line 204 using the female luer lock fitting 206 and the male luer lock fitting 208. As stated previously, the light pipe 100 may also be permanently molded into the IV line 204 during the manufacturing process. A light source 210 is coupled to the light pipe 100 via the light source socket 106. The light source 210 is preferably a self-contained unit (as shown), but may also be implemented with a central light source system. FIG. 2 illustrates how the light conductive IV system 200 utilizes a number of light pipes 100, using varying colors of light sources 210, in order to allow a user to easily distinguish between a number of prescribed fluid bags 202.

Still referring to FIG. 2, the light conductive IV system 200 of the present invention also includes an indicator panel 212 to assist a physician or other medical staff in identifying the IV lines 204 at their entry point to the patient 214. The indicator panel 212 is useful to the medical personnel so that the medical personnel can administer additional drugs through the appropriate IV line 204.

The indicator panel 212 depicted in FIG. 2 is illustrated in block diagram format, and should not limit the indicator panel 212 to the shape and size and manner of the indicator panel 212 in the figure. In fact, FIGS. 3A-3C illustrate a number of embodiments of the indicator panel 212. FIG. 3A depicts an indicator panel 212 in the form of a reflective material administered to the outside of the IV line 204, or molded directly under the surface of the IV line 204, and configured to reflect the light traveling down the IV line 204. FIG. 3B illustrates the same type of reflective material as the indicator panel 212, however, the indicator panel 212 is configured as a cross-section of the IV line 204. In other words, a reflective indicator panel 212 may be molded into the IV line 204. FIG. 3C depicts an IV line 204 having an indicator panel 212 that is a housing that encases a light conductive medium surrounding the IV line, such that the light conductive medium conducts the light to the indicator panel 212 on the outside of the housing.

The present invention has been described in terms of specific embodiments incorporating details to facilitate the understanding of the principles of construction and operation of the invention. Such reference herein to specific embodiments and details thereof is not intended to limit the scope of the claims appended hereto. It will be apparent to those skilled in the art that modifications may be made in the embodiment chosen for illustration without departing from the spirit and scope of the invention.

I claim:

1. A light pipe for conducting light from an identification luminaire to an intravenous line, the light pipe comprising:
a.) a fluid port configured to be spliced into the intravenous line, wherein the fluid port allows continuous flow of a fluid through the intravenous line;
b.) a light conducting medium configured to define the fluid port;
c.) a first fitting and a second fitting configured on a first end and a second end of the fluid port, such that the fittings effectuate connection to the intravenous line; and
d.) a light source connection socket configured to removably house the identification luminaire, and further configured to channel light from the identification luminaire into the light conducting medium, wherein the light conducting medium is configured to conduct the light into the intravenous line.

2. The light pipe as claimed in claim 1, wherein the first fitting and the second fitting are a luer fitting.

3. The light pipe as claimed in claim 1, wherein the intravenous line is coated with a translucent coating.

4. The light pipe as claimed in claim 1, wherein the light pipe is coated with a translucent coating.

5. The light pipe as claimed in claim 1, wherein the identification luminaire emits colored light.

6. The light pipe as claimed in claim 1, wherein the identification luminaire is a light emitting diode.

7. The light pipe as claimed in claim 1, further comprising an indicator panel configured on a distal end of the intravenous line, wherein the indicator panel is a reflective material, configured to reflect the light in the light conducting medium out of the light conducting medium.

8. The light pipe as claimed in claim 7, wherein the indicator panel is affixed to the outside surface of the intravenous line.

9. The light pipe as claimed in claim 7, wherein the indicator panel is embedded in the intravenous line.

10. The light pipe as claimed in claim 1, wherein the indicator panel includes:
a.) a housing configured to enclose a portion of the intravenous line; and
b.) an extension of the light conducting medium, such that the extension is in contact with the intravenous line and the housing, wherein the housing includes a transparent portion configured to allow the conducted light to leave the housing.

11. A light conductive IV system, comprising:
a.) an intravenous line configured to deliver a fluid from a fluid container to a patient;
b.) a light pipe inserted along the path of the intravenous line and including a fluid port configured to allow continuous flow of the fluid through the intravenous line, wherein a light conducting medium defines the fluid port;
c.) an identification luminaire coupled to the light pipe, wherein the light pipe channels light from the identification luminaire into the light conducting medium, wherein the light conducting medium is configured to conduct the light into the intravenous line; and
d.) an indicator panel configured on a distal end of the intravenous line, wherein the indicator panel is a reflective material, configured to reflect the light in the light conducting medium out of the light conducting medium.

12. The light conductive IV system of claim 11, further including a first fitting and a second fitting configured on a first end and a second end of the fluid port, such that the fittings effectuate connection to the intravenous lines.

13. The light conductive IV system of claim 12, wherein the first fitting and the second fitting are a luer fitting.

14. The light conductive IV system of claim 11, wherein the light pipe is molded into the intravenous line in a permanent fashion.

15. The light conductive IV system of claim 11, wherein the intravenous line and the light pipe are coated with a translucent coating.

16. The light conductive IV system of claim 11, wherein the identification luminaire emits colored light, and further wherein when the system includes a plurality of intravenous lines, a plurality of identification luminaires are utilized, each identification luminaire emitting a different color light.

17. The light conductive IV system of claim 11, wherein the identification luminaire is a light emitting diode.

18. The light conductive IV system of claim 11, wherein the indicator panel is affixed to the outside surface of the intravenous line.

19. The light conductive IV system of claim 11, wherein the indicator panel is embedded in the intravenous line.

20. A light pipe comprising:
a.) a fluid port configured to be spliced into an intravenous line, wherein the fluid port allows continuous flow of a fluid through the intravenous line;
b.) a light conducting medium configured to define the fluid port, and further configured to allow continuous light conduction through the intravenous line;
c.) a first luer fitting and a second luer fitting configured on a first end and a second end of the fluid port, such that the luer fittings effectuate connection to the intravenous line; and
d.) a light source connection socket configured to removably house an identification luminaire, and further configured to channel light from the identification luminaire into the light conducting medium, wherein the light conducting medium is configured to conduct the light into the intravenous line, and further wherein the light pipe is coated with a translucent coating.
